(19) United States
(12) Patent Application Publication
(10) Pub. No.: US 2024/0164723 A1
(43) Pub. Date: May 23, 2024

(54) LOW-NOISE SENSOR SYSTEM

(71) Applicant: Cercacor Laboratories, Inc., Irvine, CA (US)

(72) Inventors: Cristiano Dalvi, Lake Forest, CA (US); Jeroen Poeze, Rancho Santa Margarita, CA (US)

(21) Appl. No.: 18/521,712

(22) Filed: Nov. 28, 2023

Related U.S. Application Data
(63) Continuation of application No. 18/045,970, filed on Oct. 12, 2022, now Pat. No. 11,864,922.
Application No. 18/045,970 is a continuation of application No. 16/831,497, filed on Mar. 26, 2020, now Pat. No. 11,504,066.
Application No. 16/831,497 is a continuation of application No. 15/257,892, filed on Sep. 6, 2016, now abandoned.
(60) Provisional application No. 62/214,440, filed on Sep. 4, 2015.

(51) Int. Cl. A61B 5/00; A61B 5/026; A61B 5/1455; A61B 5/1491

(52) U.S. Cl. CPC A61B 5/7203; A61B 5/0261; A61B 5/14552; A61B 5/1491; A61B 5/7278; A61B 2560/0214; A61B 2562/185; A61B 2562/222; A61B 2562/227

(57) ABSTRACT

A sensor system has a low-noise sensor controller providing communications between an active-temperature-regulated optical sensor and an external monitor. A low-noise sensor controller drives optical emitters, receives resulting detected signals after attenuation by a blood perfused tissue site and communicates the detector signals to the attached signal processor. An optically-isolated controller front-end receives and digitizes the detected signals. A controller serializer transmits the digitized detector signal to the processor via a single, shielded coaxial cable.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application is a continuation of U.S. patent application Ser. No. 18/045,970, filed Oct. 12, 2022, titled “LOW-NOISE SENSOR SYSTEM,” which is a continuation of U.S. patent application Ser. No. 16/831,497, filed Mar. 26, 2020, titled “LOW-NOISE SENSOR SYSTEM”, which is a continuation of U.S. patent application Ser. No. 15/257,892, filed Sep. 6, 2016, titled “LOW-NOISE SENSOR SYSTEM”, which claims priority benefit under 35 U.S.C. § 119(e) to U.S. Provisional Patent Application Ser. No. 62/214,440 titled “LOW-NOISE SENSOR SYSTEM”, filed Sep. 4, 2015, each of which are hereby incorporated by reference herein in their entirety.

### INCORPORATION BY REFERENCE OF RELATED CASES

The present disclosure is generally related to U.S. Pat. No. 8,688,183 titled Emitter Driver for Noninvasive Patient Monitor, filed Sep. 2, 2010 and issued Apr. 1, 2014; U.S. Pat. No. 8,225,027 titled Multiple Wavelength Sensor Substrate, filed Jul. 19, 2010 and issued Aug. 28, 2012; U.S. Pat. No. 8,630,691 titled Multi-Stream Sensor Front Ends For Noninvasive Measurement of Blood Constituents, filed Aug. 3, 2009 and issued Jan. 14, 2014; and U.S. Pat. No. 8,688,183 titled Emitter Driver For Noninvasive Patient Monitor, filed Sep. 2, 2010 and issued Apr. 1, 2014. Each of the above-referenced issued patents are hereby incorporated in their entireties by reference herein.

### BACKGROUND OF THE INVENTION

Noninvasive physiological monitoring systems for measuring constituents of circulating blood have advanced from basic pulse oximeters capable of measuring blood oxygen saturation to advanced blood parameter monitors capable of measuring various blood constituents. A basic pulse oximeter typically includes an optical sensor, a monitor for processing sensor signals and displaying results and a cable electrically interconnecting the sensor and the monitor. A basic pulse oximetry sensor typically has a red wavelength light emitting diode (LED), an infrared (IR) wavelength LED and a photodiode detector. The LEDs and detector are attached to a patient tissue site, such as a finger. The cable transmits drive signals from the monitor to the LEDs, and the LEDs respond to the drive signals to transmit light into the tissue site. The detector generates a photoplethysmograph signal responsive to the emitted light after attenuation by pulsatile blood flow within the tissue site. The cable transmits the detector signal to the monitor, which processes the signal to provide a numerical readout of oxygen saturation (SpO2) and pulse rate, along with an audible pulse indication of the person's pulse. The photo-plethysmograph waveform may also be displayed.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entirety by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entirety by reference herein. Advanced pulse oximetry systems including Masimo SET® low noise optical sensors and read through motion pulse oximetry monitors for measuring SpO2, pulse rate (PR) and perfusion index (Pl) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are capable of measuring various blood parameters in addition to SpO2, such as total hemoglobin and carboxyhemoglobin to name a few. Advanced blood parameters measurement systems are described in at least U.S. Pat. No. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. No. 7,957,780, filed Mar. 1, 2006, titled Physiological Parameter Confidence Measure and U.S. Pat. No. 8,224,411, filed Mar. 1, 2006, titled Noninvasive Multi-Parameter Patient Monitor, all assigned to Cercacor Laboratories, Inc., Irvine, CA (“Cercacor”) and all incorporated in their entirety by reference herein. An advanced parameter measurement system that includes acoustic monitoring is described in U.S. Pat. Pub. No. 2010/027 4099, filed Dec. 21, 2009, titled Acoustic Sensor Assembly, assigned to Masimo and incorporated in its entirety by reference herein.

Advanced blood parameter measurement systems include Masimo Rainbow® SET, which provides measurements in addition to SpO2, such as total hemoglobin (SpHb™), oxygen content (SpOC™), methemoglobin (SpMet®), carboxyhemoglobin (SpCO®) and PVI®. Advanced blood parameter sensors include Masi mo Rainbow® adhesive, ReSposable™ and reusable sensors. Advanced blood parameter monitors include Masimo Radical-7™, Rad-87™ and Rad-57™ monitors, all available from Masimo. Advanced parameter measurement systems may also include acoustic monitoring such as acoustic respiration rate (RRa™) using a Rainbow Acoustic Sensor™ and Rad-87™ monitor, available from Masimo. Such advanced pulse oximeters, low noise sensors and advanced parameter systems have gained rapid acceptance in a wide variety of medical applications, including surgical wards, intensive care and neonatal units, general wards, home care, physical training, and virtually all types of monitoring scenarios.

### SUMMARY OF THE INVENTION

A low-noise sensor system has a low-noise sensor controller providing communications between an active-temperature-regulated optical sensor and a signal processor. The low-noise sensor controller drives optical emitters, receives resulting detected signals after attenuation by a blood perfused tissue site and communicates those signals to the attached signal processor. Advantageously, all controller-received sensor data is serially-communicated to the processor via a single, shielded coaxial cable.

One aspect of a low noise sensor system is an optical sensor having emitters for transmitting optical radiation into a blood-perfused tissue site and detectors responsive to optical radiation after attenuation by pulsatile blood flow within the tissue site so as to generate sensor signals. A sensor controller has an optically-isolated front-end that receives and digitizes the sensor signals. A serializer is in communications with a single-conductor, shielded cable so as to serially transmit the digitized sensor signals to an external monitor for deriving bloodrelated physiological parameters responsive to detector signals from the detector.

Another aspect of a low noise sensor processor is an optically and physically isolated front-end portion of the sensor controller that receives detected sensor signals and serially communicates the sensor data derived from those signals to a signal processor. The serial data is transmitted to the signal processor over a single coaxial cable. Another aspect of a low noise sensor processor is active temperature control of the sensor emitters and detectors via independent thermoelectric (TEC) devices on each of the emitter assembly and detector assembly. A further aspect of a low noise sensor processor is the sensor controller is battery powered, with the battery being charged via a relay isolated DC power supply during off cycles.

Further aspects of a low-noise sensor system include an optical sensor having emitters for transmitting optical radiation into a blood-perfused tissue site and detectors responsive to optical radiation after attenuation by pulsatile blood flow within the tissue site so as to generate sensor signals. A sensor controller has an optically-isolated front-end that receives and digitizes the sensor signals. A serializer is in communications with a shielded conductor so as to serially transmit the digitized sensor signals to an external monitor for deriving blood-related physiological parameters responsive to detector signals from the detector. A DC power supply includes a battery, a sensor-controller power output and a relay that connects the DC power supply to the battery in a recharge mode and that connects the battery to the sensor controller power output in an operational mode.

In an embodiment, the interface between the sensor controller and the external monitor consists solely of three conductors including a DC power supply plus (+) conductor, a DC power supply minus (−) conductor and the shielded conductor. In an embodiment, a first thermoelectric cooler is in thermodynamic communications with the emitters, a second thermoelectric cooler is in thermodynamic communications with the detectors and a temperature regulator controls the thermoelectric coolers so as to maintain the emitters and detectors at predetermined temperatures. In another embodiment, sensor status is transmitted to the external monitor with the sensor data, a micro-controller sequentially activates the emitters and controls emitter drive currents, and the optically-isolated front end further comprises a programmable gain amplifier that individually amplifies detector signals and an analog-to-digital converter (ADC) that digitizes the detector signals for transmission to the monitor.

An additional aspect of a low-noise sensor is attaching an optical sensor to a tissue site, emitting multiple wavelengths of optical radiation into the tissue site, detecting optical radiation after attenuation by pulsatile blood flow within the tissue site, digitizing the detected optical radiation, serially transmitting the digitized detected optical radiation to a monitor and calculating physiological parameters based upon the digitized detected optical radiation. In an embodiment, emitters and detectors are independently thermo-electrically cooled.

In various embodiments, a sensor controller controls the digitizing and serial transmitting, powers the sensor controller with a battery and intermittently charges the battery when not digitizing and serial transmitting. A 3-wire interface is provided between the sensor controller and an external monitor including a 2-wire interface for the charging and a 1-wire interface for the serial transmitting. Sensor status is serialized and combined with sensor data for transmission to the monitor. A microcontroller controls an emitter driver sequence and an emitter drive current. The sensor controller is optically isolated.

In a yet a further embodiment, a low noise sensor apparatus has an emitter means for illuminating a tissue site, a detector means for receiving tissue site illumination after tissue site absorption and generating a detector signal in response, a front-end means for amplifying and digitizing the detector signal and a serializer means for communicating the amplified and digitized detector signal to an external monitor. In an aspect, a sensor status means is for communicating sensor temperatures and accelerations to the external monitor via the serializer means. A temperature regulating means is for communications with the emitter means and the detector means. A rechargeable battery powered means provides low-noise power to at least the front-end means and serializer means. An optical isolation means reduces noise for the front-end means. A three-wire isolation means is disposed between at least the front-end means and the external monitor for reducing noise.

### BRIEF DESCRIPTION OF THE DRAWINGS

FIG. 1 is a general illustration of a low-noise sensor system including a monitor, an optical sensor and a low-noise sensor controller disposed between the monitor and optical sensor;

FIG. 2 is a detailed illustration of a low-noise sensor system illustrating a low-noise sensor controller providing active-temperature-regulation of the sensor emitters and detectors and a low-noise, serial communications with the monitor via a single, shielded coaxial cable;

FIG. 3 is a general block diagram of a low-noise sensor controller providing active-temperature-regulation of an optical sensor, an optically-isolated front-end to the sensor detectors and shielded, serial communications to the monitor signal processor;

FIG. 4 is a general block diagram of low-noise sensor controller electrical communications with an optical sensor;

FIG. 5 is a detailed block diagram of low-noise sensor controller drive signals to optical sensor emitters;

FIG. 6 is a detailed block diagram of an opto-isolated sensor front-end receiving and digitizing detector signals for low-noise transmission to the monitor;

FIG. 7 are block diagrams of a serializer interface for transmitting sensor data and sensor status to a monitor and a micro-controller interface to the sensor temperature regulators;

FIG. 8 is a detailed block diagram of micro-controller control and drive signals to both optical sensor emitters and sensor temperature regulators; and

FIG. 9 is a detailed block diagram of a coaxial-cable serial data interface between the low-noise sensor controller and the monitor and a relay-isolated, rechargeable-battery power supply for the low-noise sensor controller.

### DETAILED DESCRIPTION OF THE PREFERRED EMBODIMENT

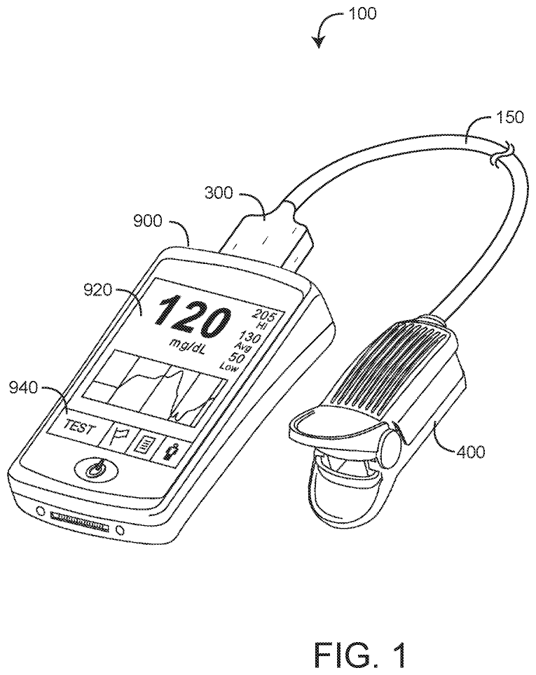

FIG. 1 generally illustrates a low-noise sensor system 100 including a monitor 900, an optical sensor 400 and a low-noise sensor controller 300. In various embodiments, the low-noise sensor controller 300 may be incorporated within or immediately adjacent to the monitor 900, incorporated within or immediately adjacent to the sensor 400, incorporated between cable portions interconnecting the monitor 900 and the optical sensor 400 or incorporated within or adjacent a cable connector, as shown. The low-noise sensor controller 300 drives optical emitters, receives resulting detected signals after attenuation by a blood perfused tissue site and communicates those signals to the monitor 900 signal processor. Advantageously, sensor data is serially-communicated to an external monitor. In an embodiment, the serial communications is via a single, shielded line, such as a coaxial cable. In an embodiment, the serial communications is a high-speed optical data link.

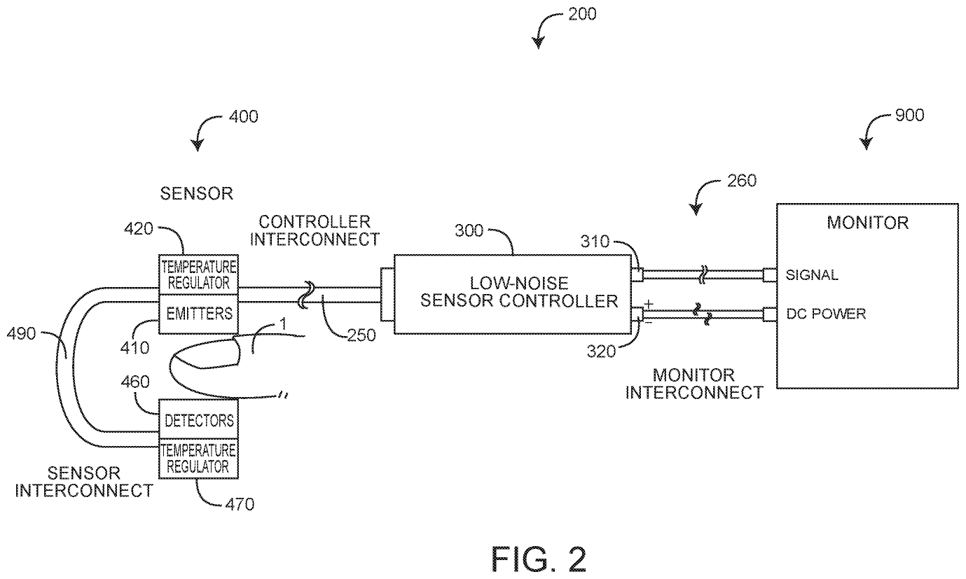

FIG. 2 generally illustrates a low-noise sensor system 200 embodiment having an optical sensor 400, a monitor 900 and a low-noise sensor controller 300 providing communication between the sensor 400 and the monitor 900. The lownoise sensor controller 300 provides active-temperature-regulation of sensor emitters 410 and detectors 460 and low-noise, serial communications with the monitor 900 via a single, shielded coaxial cable 310. An attachment mechanism (not shown), such as adhesive pads or a finger clip (400 FIG. 1) positions the emitters 410 and detectors 460 relative to a blood perfused tissue site 1, such as a fingernail bed. The emitters 410 are driven by the controller 300 via a controller interconnect 250 so as to transmit light of various wavelengths into the tissue site 1. The detectors 460 receive the emitted light after attenuation by the blood perfused tissue so as to return a corresponding sensor signal to the controller 300 via the interconnect 250. In an embodiment, the controller interconnect 250 is a flex circuit or cable.

As shown in FIG. 2, in an embodiment, an emitter temperature regulator 420 is in thermal communication with the emitters 410 and a detector temperature regulator 470 is in thermal communication with the detectors 460 so as to independently control emitter and detector temperatures. Advantageously, emitter temperature regulation 420 precisely maintains emitters 410 at pre-specified emitter wavelengths. Likewise, detector temperature regulation 470 precisely maintains detectors 460 at pre-specified responsivity to those wavelengths.

Also shown in FIG. 2, the sensor 400 may be placed on various tissue site locations. Emitters 410 and detectors 460 may be positioned on the same side of a tissue site 1 in a reflective sensor position or may be positioned on opposite sides of a tissue site 1 in a transmissive sensor position as shown. Transmissive sites may include a fingertip as shown, an ear lobe or feet, to name a few. Reflective sites may include a forehead, a chest, arms, legs, hands or feet, to name a few. A sensor interconnect 490 provides electrical communications between sensor components 410, 420, 460, 470 and the controller 300 via the controller interconnect 250. The sensor controller 300 provides communications between the sensor 400 and a monitor 900.

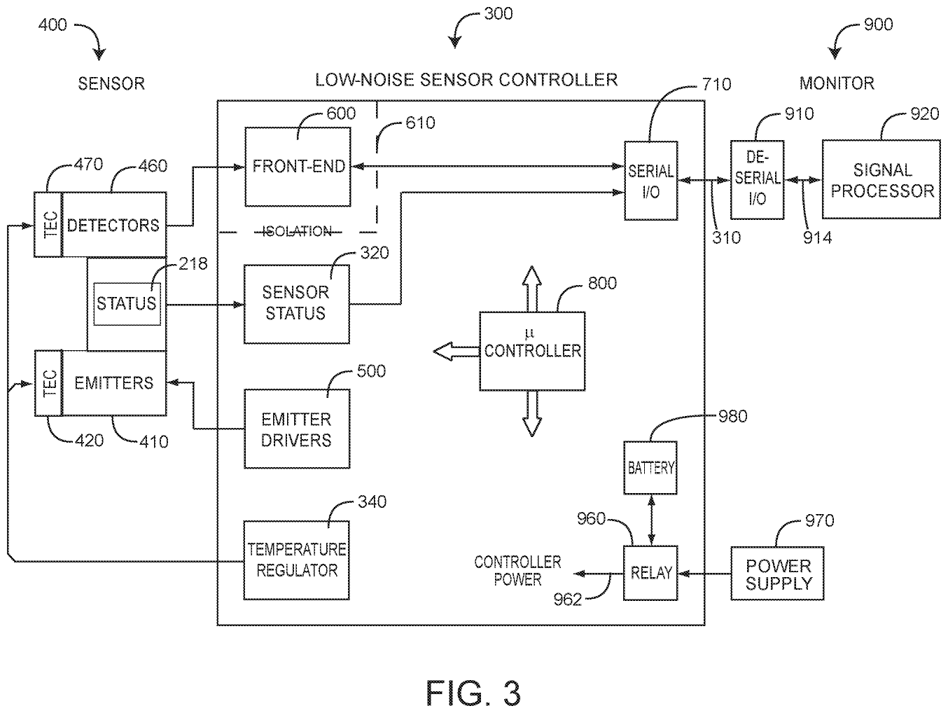

FIG. 3 further illustrates a low-noise sensor controller 300 embodiment providing electrical communications between an active-temperature-regulated optical sensor 400 and a signal processor 920. The sensor controller 300 advantageously has a sensor front-end 600 that is physically segregated and optically isolated 610 from the rest of the sensor controller components. Further, sensor data is advantageously serially-communicated from the sensor front-end 600 to the monitor 900 via a single, shielded coaxial cable 310. In addition, the sensor controller 300 has a temperature regulator 340 that provides active temperature control 420, 470 to the sensor emitters 410 and detectors 460. Also, the sensor controller power 962 is battery-supplied 980 during operation. Relay switching 960 provides intermittent connections to a DC power supply 970 for recharging the battery 980 when the sensor controller 300 is idle. Sensor detectors 460 communicate with a sensor controller front-end 600, which is physically- and optically-isolated 610 from other portions of the sensor controller 300.

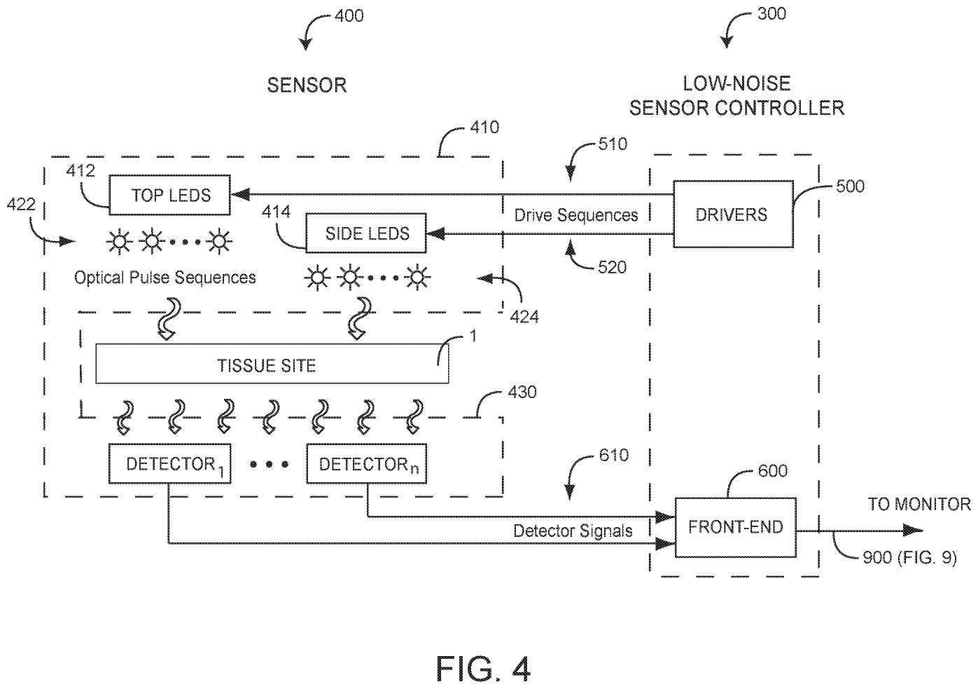

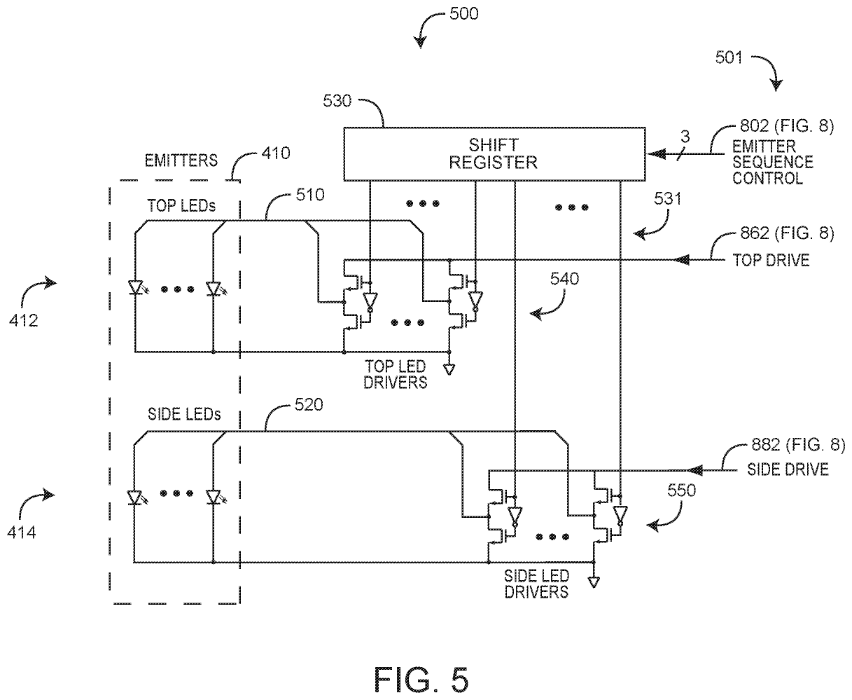

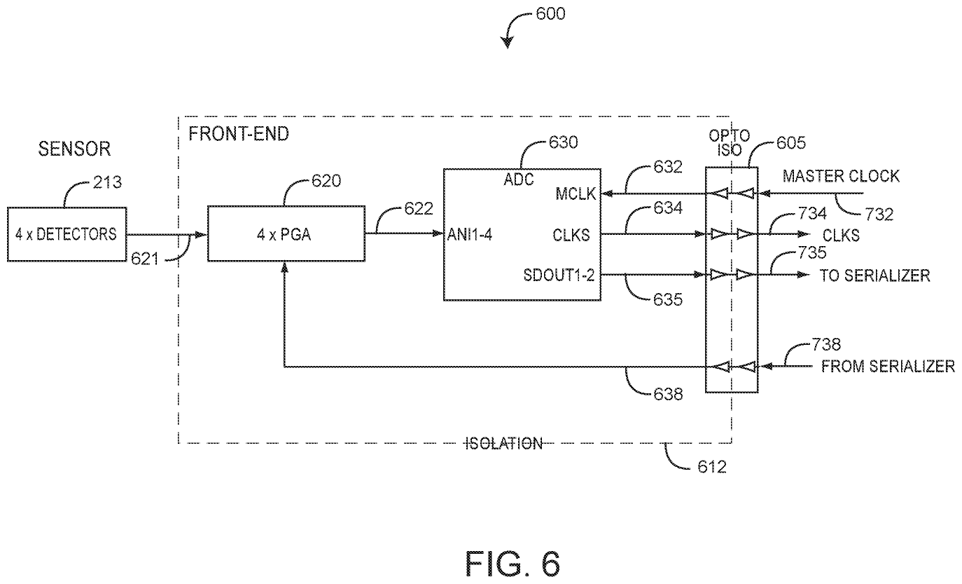

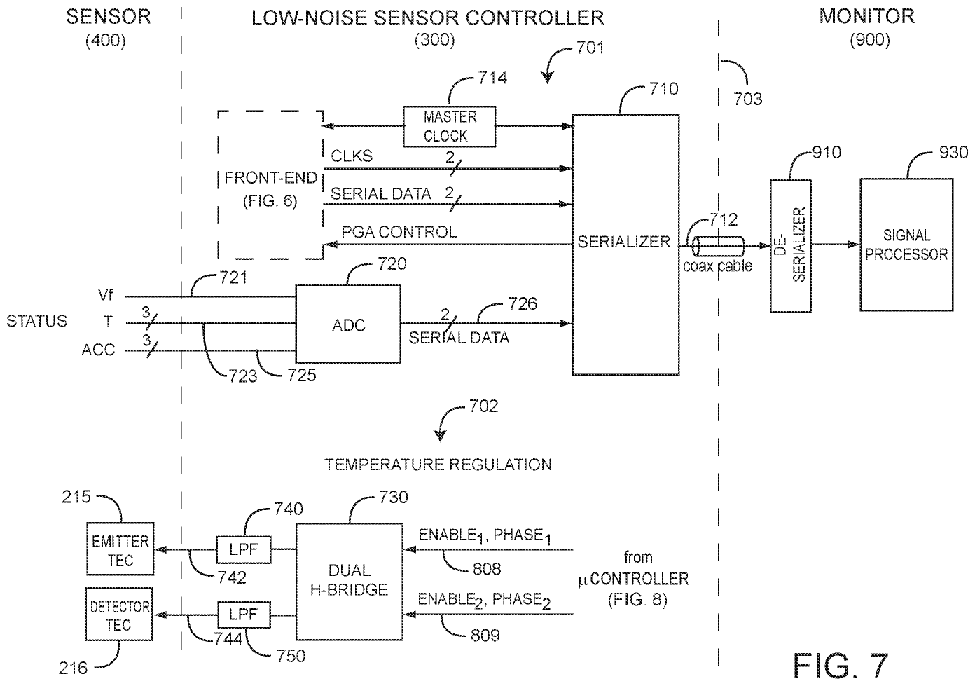

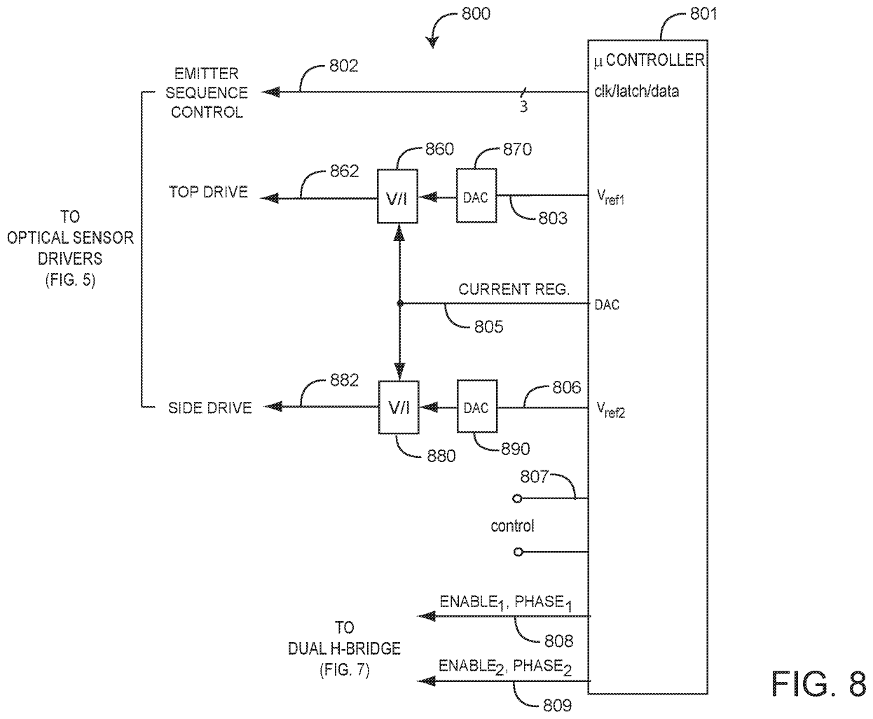

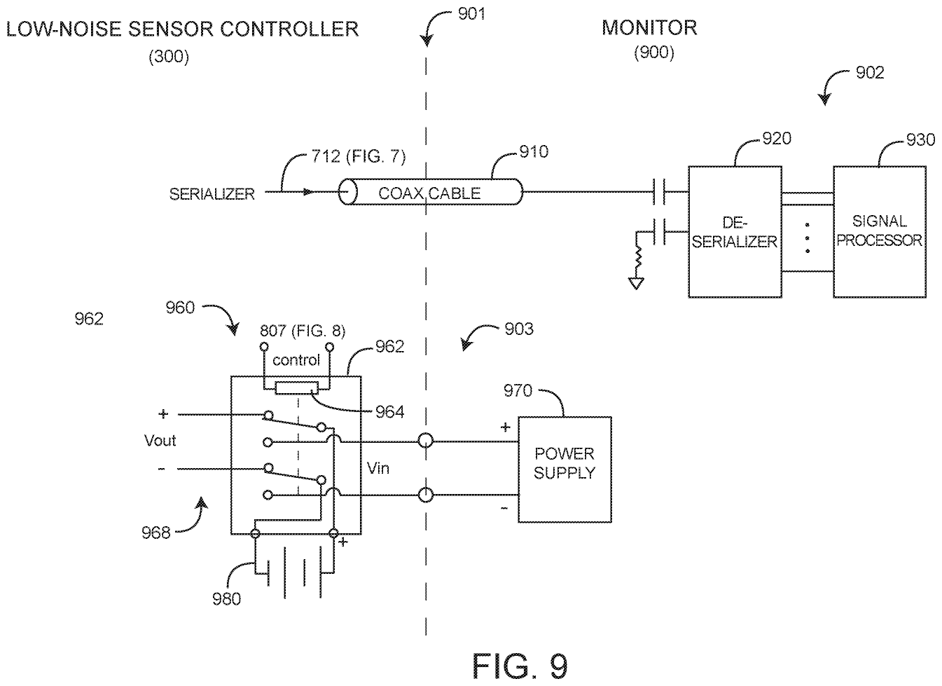

These serial data, battery operated, opto-isolated sensor front-end and temperature-controlled emitter/detector features are described in further detail with respect to FIGS. 4-9, below. Emitter drivers 500 are generally described with respect to FIG. 4 and specifically described with respect to FIG. 5. Serial signal processor communications and active sensor temperature control are described in detail with respect to FIG. 5. The opto-isolated sensor 600 front-end is described in detail with respect to FIG. 6. Sensor status 320 and sensor temperature regulation 340 are described in detail with respect to FIG. 7. Microcontroller control 800 of emitter drivers 500 and of sensor temperature control 340 are described in detail with respect to FIG. 8. Serial data communications 710, switchable battery-powered operation 980 and relay isolated recharging 960, 970 are described in further detail with respect to FIG. 9.

FIG. 4 illustrates an optical sensor 400 and its corresponding interface 510, 520, 610 to LED drivers 500 and a front-end 600 of the low-noise sensor controller 300. The sensor 400 has light-emitting diodes (LEDs) 410 that illuminate a tissue site 1 in response to LED drive signals 510, 520 that sequentially activate LEDs 412, 414. Detectors 430 generate detector signals 610 to the front-end 600 responsive to the tissue site illumination 422, 424 after absorption by pulsatile blood flow within the tissue site 1.

As shown in FIG. 4, the sensor controller 300 has LED drivers 500 that sequentially activate top LEDs 412 and side LEDs 414 so as to illuminate the tissue site 1 with multiple wavelengths of light in a predetermined wavelength sequence. The term “top LEDs” refers to light emitting diodes that emit light perpendicular to the plane of their p-n junction (top emission), and the term “side LEDs” refers to light emitting diodes that emit light parallel to the plane of their p-n junction (side emission).

Also shown in FIG. 4, the sensor controller 300 also has a front-end 600 that transmits binary data to the monitor 900 (FIG. 9) that is responsive to detector signals 610. In particular the LEDs 412, 414 each generate a predetermine sequence of optical pulses 422, 424 at specific optical wavelengths so that the detectors signals 610 are sequentially responsive to tissue site absorption at each optical wavelength. The front-end 600 transmits this information to the monitor 900 (FIG. 9) for analysis of tissue site blood flow constituents.

FIG. 5 further illustrates emitter drive signals 501 originating from the sensor controller 300 (FIG. 3) microcontroller 800 (FIG. 8). Advantageously, there are independent top LED drivers 540 and side LED drivers 550. In this manner, the microcontroller circuitry 800 (FIG. 8) can independently set voltage references for top LEDs 412 and side LEDs 414. Emitter control 802 (FIG. 8) includes clock, latch and data signals for loading an enable bit into a shift register 530 and then clocking that enable bit across each of the driver enable lines 531. The top LED drive currents are set by a microcontroller top drive reference 862 (FIG. 8) and the side LED currents drive currents are set by a microcontroller side drive reference 882 (FIG. 8).

FIG. 6 illustrates an isolated sensor front-end 600 having four detector inputs 621 corresponding to the sensor's four detectors 213. Advantageously, the front-end 600 is physically and optically isolated from other sensor controller 300 (FIG. 3) circuits and components. Specifically, there are no conductive signal paths between the front-end 600 and the remainder of the sensor controller 300 (FIG. 3), as all signals to or from the front-end 600 are optically-communicated 605. As such, the sensor's detector signals 621 have a relatively high signal-to-noise ratio as transmitted to the serializer 635, 735.

As shown in FIG. 6, the detector inputs 621 are individually amplified by four programmable-gain amplifiers (PGA) 620, which provide four inputs 622 to an analog-to digital converter (ADC) 630. These inputs 622 are digitized 630 into two serial data outputs 635, which are transmitted across an opto-isolator interface 612 as isolated outputs 735 to the serializer 710 (FIG. 7). A master clock 714 (FIG. 7) provides an isolated clock input 632, 732 to the ADC 630, which generates synchronized clocks 634 corresponding to the serial data outputs 635.

FIG. 7 illustrates additional sensor controller 300 functions, components and interfaces including serializer inputs 701, sensor temperature regulation 702 and the serializer/monitor interface 703. The serializer 710 receives clocks and clock-sync'd serial data inputs 701 from the front end (FIG. 6). The serializer 710 also provides a programmable gain control (PGA) 701; 638, 738 (FIG. 6) to the front-end PGA 620 (FIG. 6). A sensor status analog-to-digital converter (ADC) 720 provides emitter, detector and finger temperatures 723 and 3-axis sensor accelerometer data 725 to the monitor 900 via the serializer 710. Also shown in FIG. 7, the microcontroller 801 (FIG. 8) provides dual enable and phase controls 808, 809 to the dual H-bridge 730 for dual temperature control signals 742 744 to the sensor temperature regulators 215, 216.

FIG. 8 illustrates micro-controller control and drive signals 800 to both optical sensor emitters and sensor temperature regulators. In one aspect, a microcontroller 801 provides sequence control 802 for selectively activating the sensor emitters (LEDs) 410 (FIG. 5) one at a time. The micro-controller 801 also provides specifically regulated drive currents 862, 882 for each of the LEDs. Digital voltage references 803, 806 are digital-to-analog converted 870, 890 to analog current references by voltage to current (V/I) converters 860, 880. In turn, the V/l's 860, 880 generate drive currents 862, 882 for activating the LEDs 412, 414 (FIG. 5). Also shown in FIG. 8, the micro-controller 801 provides enable and phase control 808, 809 to the H-bridge 730 (FIG. 7) for emitter and detector temperature regulation.

FIG. 9 illustrates an advantageous three-wire interface 901 between the low-noise sensor controller 300 and the monitor 900 for data serialization/deserialization and battery power supply recharging. In particular, this interface 901 includes an advantageous single wire, serial data interface between the low-noise sensor controller 300 serializer output 712 (FIG. 7) to the monitor 900. This interface 901 also includes an advantageous two-wire interface 903 between a monitor-side power supply 970 and a relay-isolated, rechargeable-battery power supply 980 that powers the low-noise sensor controller 300. The serial data interface is a coaxial cable 910 that inputs to a de-serializer 920, which provides corresponding sensor data and status to the monitor signal processor 930. The power interface 903 includes two wires from the monitor-side power supply 970 that recharge the low-noise sensor-controller 300 relay-isolated, rechargeable-battery power supply 960. The rechargeable power supply 960 has a relay 962, a relay control 964, a power output 968 and a rechargeable battery 980.

A low noise sensor system has been disclosed in detail in connection with various embodiments. These embodiments are disclosed by way of examples only and are not to be construed as limiting the scope of the claims that follow. One of ordinary skill in art will appreciate many variations and modifications.

1. (canceled)

2. A low-noise sensor system comprising:
a housing comprising:
an optical sensor configured to detect optical radiation after attenuation by blood at a tissue site so as to generate sensor signals;
a sensor controller having an optically-isolated portion that receives and digitizes the sensor signals;
a serializer in communications with a shielded conductor so as to serially transmit the digitized sensor signals to one or more hardware processors to derive one or more physiological parameters based on the sensor signals; and
a display configured to display the one or more physiological parameters.

3. The low-noise sensor system according to claim 2 further comprising:
a DC power supply;
a battery;
a sensor-controller power output; and
a relay that connects the DC power supply to the battery in a recharge mode and that connects the battery to the sensor-controller power output in an operational mode.

4. The low-noise sensor system of claim 2 wherein the one or more hardware processors are configured to cause display of the one or more physiological parameters on a display.

5. The low-noise sensor system according to claim 2 wherein an interface between the sensor controller and the one or more hardware processors consists solely of three conductors including a DC power supply plus (+) conductor, a DC power supply minus (−) conductor and the shielded conductor.

6. The low-noise sensor system according to claim 4, wherein the optical sensor comprises emitters and detectors and wherein the housing further comprises:
a first thermoelectric cooler in thermodynamic communications with the emitters;
a second thermoelectric cooler in thermodynamic communications with the detectors; and
a temperature regulator that controls the thermoelectric coolers so as to maintain the emitters and detectors at predetermined temperatures.

7. The low-noise sensor system according to claim 6 wherein a sensor status is transmitted to the one or more hardware processors with sensor data through the interface.

8. The low-noise sensor system according to claim 7 further comprising a micro-controller that sequentially activates the emitters and controls emitter drive currents.

9. The low-noise sensor system according to claim 7 wherein the optically-isolated portion further comprises:
a programmable gain amplifier that individually amplifies detector signals; and
an analog-to-digital converter (ADC) that digitizes the detector signals for transmission to the one or more hardware processors.
